(19) United States
(12) Patent Application Publication
(10) Pub. No.: US 2011/0245541 A1
(43) Pub. Date: Oct. 6, 2011

(54) Charge Transport Molecules And Method For Preparing Same

(75) Inventor: Yuhua Tong, Webster, NY (US)

(73) Assignee: XEROX CORPORATION, Norwalk, CT (US)

(21) Appl. No.: 12/752,602

(22) Filed: Apr. 1, 2010

(51) Int. Cl. C07C 217/84; G03G 5/04

(52) U.S. Cl. 564/443; 430/58.75; 399/130

(57) ABSTRACT

A compound of the formula

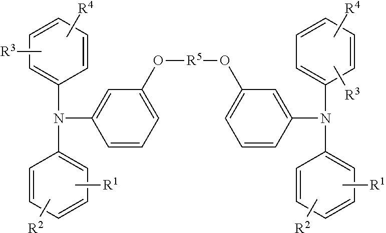

wherein R¹, R², R³, and R⁴ can be the same or different, and wherein each of R¹, R², R³, and R⁴ are independently selected from (i) hydrogen, (ii) an alkyl group, (iii) an aryl group, (iv) an arylalkyl group, (v) an alkylaryl group, (vi) an alkoxy group, (vii) an aryloxy group, (viii) an arylalkyloxy group, (ix) an alkylaryloxy group; and wherein R⁵ is (i) an alkylene group, (ii) an arylene group, which can be substituted or unsubstituted arylene, and wherein hetero atoms may optionally be present in the arylene group, (iii) an arylalkylene group, or (iv) an alkylarylene group. Further, a process for preparing a charge transport compound comprises contacting a hydroxy-functionalized triarylamine with a dihalide in an alkaline-water solution at room temperature; wherein the raw material for the dihalide comprises a purified or recovered halogen-organic solvent-containing waste stream.

### TECHNICAL FIELD

Described herein are charge transport molecules for imaging devices such as organic photoreceptors. More particularly, described herein are charge transport molecules and a method for preparing charge transport molecules that is environmentally friendly and provides the ability to convert organic halide waste into valuable charge transport materials.

### BACKGROUND

The present disclosure is generally related to imaging members and more particularly related to photosensitive members and in embodiments to charge transport molecules for imaging members and methods for preparing same. In embodiments, a compound of the formula

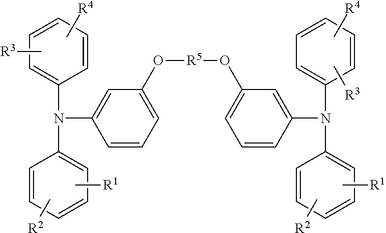

is disclosed wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group; and wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group.

In the art of electrophotography, an electrophotographic plate comprising a photoconductive insulating layer on a conductive layer is imaged by first uniformly electrostatically charging the surface of the photoconductive insulating layer. The plate is then exposed to a pattern of activating electromagnetic radiation such as light, which selectively dissipates the charge in the illuminated areas of the photoconductive insulating layer while leaving behind an electrostatic latent image in the non-illuminated areas. This electrostatic latent image may then be developed to form a visible image by depositing finely divided electroscopic toner particles on the surface of the photoconductive insulating layer. The resulting visible toner image can be transferred to a suitable receiving member such as paper. This imaging process may be repeated many times with reusable photoconductive insulating layers.

Electrophotographic imaging members are usually multilayered photoreceptors that comprise a substrate support, an electrically conductive layer, an optional hole blocking layer, an adhesive layer, a charge generating layer, and a charge transport layer in either a flexible belt form or a rigid drum configuration. Multilayered flexible photoreceptor belts may include an anti-curl layer on the backside of the substrate support, opposite to the side of the electrically active layers, to render the desired photoreceptor flatness. One type of multilayered photoreceptor comprises a layer of finely divided particles of a photoconductive inorganic or organic compound dispersed in an electrically insulating organic resin binder. The charge generating layer is capable of photogenerating holes and injecting the photogenerated holes into the charge transport layer. Photoreceptors can also be single layer devices. For example, single layer organic photoreceptors typically comprise a photogenerating pigment, a thermoplastic binder, and hole and electron transport materials.

U.S. Pat. No. 4,265,990, which is hereby incorporated by reference herein in its entirety, discloses a layered photoreceptor having a separate charge generating (photogenerating) layer (CGL) and charge transport layer (CTL). The charge generating layer is capable of photogenerating holes and injecting the photogenerated holes into the charge transport layer. The photogenerating layer utilized in multilayered photoreceptors include, for example, inorganic photoconductive particles or organic photoconductive particles dispersed in a film forming polymeric binder. Inorganic or organic photoconductive materials may be formed as a continuous, homogeneous photogenerating layer.

Examples of photosensitive members having at least two electrically operative layers including a charge generating layer and diamine containing transport layer are disclosed in U.S. Pat. Nos. 4,265,990; 4,233,384; 4,306,008; 4,299,897; and 4,439,507, the disclosures of each of which are hereby incorporated by reference herein in their entireties.

Charge transport layers are known to be comprised of any of several different types of polymer binders that have a charge transport material dispersed therein. The charge transport layer can contain an active aromatic diamine small molecule charge transport compound dissolved or molecularly dispersed in a film forming binder. This type of charge transport layer is described, for example, in U.S. Pat. No. 4,265,990, the disclosure of which is incorporated by reference herein in its entirety. Although excellent toner images can be obtained with such multilayered photoreceptors, it has been found that when high concentrations of active aromatic diamine small molecule charge transport compound are dissolved or molecularly dispersed in a film forming binder, the small molecules tend to crystallize with time under conditions such as higher machine operating temperatures, mechanical stress or exposure to chemical vapors. Such crystallization can cause undesirable changes in the electro-optical properties, such as residual potential build-up which can cause cycle-up. Moreover, the ranges of binders and binder solvent types available for use during coating operations is limited when high concentrations of the small molecules are sought for the charge transport layer.

Another type of charge transport layer has been described which uses a charge transport polymer. This type of charge transport polymer includes, but is not limited to, materials such as poly-N-vinyl carbazole, polysilylenes, and others. Other charge transporting materials include polymeric arylamine compounds and related polymers. Charge transport layer materials such as these are described in U.S. Pat. Nos. 4,801,517; 4,806,443; 4,806,444; 4,818,650; 4,871,634; 4,935,487; 4,937,165; 4,956,440; 4,959,288; 5,030,532; 5,155,200; 5,262,512; 5,306,586; 5,342,716; 5,356,743; 5,413,886; 5,639,581; 5,770,339; and 5,814,426; the disclosures of each of which are incorporated by reference herein in there entireties.

The appropriate components and process aspects of the each of the foregoing U.S. patents may be selected for the present disclosure in embodiments thereof.

Charge transport molecules are a critical component of organic photoreceptors. Higher charge transport mobility is desired for the application of these materials. Generally, charge transport materials comprise triarylamines and their derivatives which are synthesized by the coupling reaction of diarylamines and aryl halides under inert atmosphere at high temperature. Halogen-containing organic solvents such as methylene chloride are commonly used in the fabrication of photoreceptors. The recovery of halo-organic solvent waste is a critical environmental problem in the chemical industry.

What is needed in the art is a process for preparing charge transport materials that is environmentally friendly and convenient. What is further needed is a process for preparing charge transport materials that does not require expensive or complicated reaction protocols. What is further needed is a process that produces charge transport materials having desired high charge transport mobility.

### SUMMARY

Described is a compound of the formula

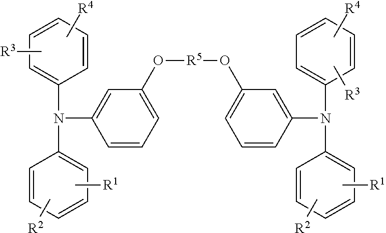

wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group; and

wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group.

Also described is a process for preparing a charge transport compound comprising contacting a hydroxy-functionalized triarylamine of the formula

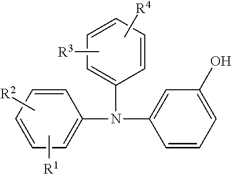

with a dihalide of the formula

R⁵X₂

wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group;

wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group;

wherein X is a halogen;

in an alkaline-water solution at room temperature; and

optionally, treating the product to provide a purified product.

Further described is an imaging member comprising a substrate; thereover a charge generating layer; and thereover a charge transport layer comprising a compound of the formula

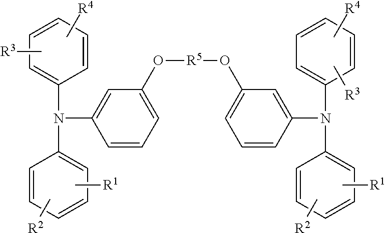

wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group; and wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group.

### BRIEF DESCRIPTION OF THE DRAWINGS

FIG. 1 is a graph showing mobility (in cm²V⁻¹S⁻¹) (y-axis) versus field (V/cm) (x-axis) for the several embodiments of the present disclosure.

### DETAILED DESCRIPTION

A charge transport molecule is described comprising a compound of the formula

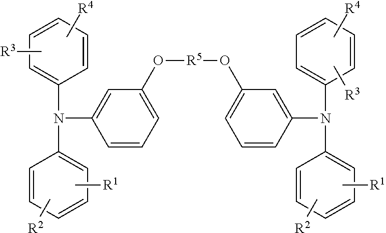

wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms such as oxygen, nitrogen, sulfur, silicon, phosphorus, boron, and the like, may optionally be present in the alky group, in embodiments, having from about 1 to about 20 carbon atoms, or from about 1 to about 18 carbon atoms, although the numbers can be outside of these ranges, (iii) an aryl group, which can be substituted or unsubstituted aryl, and wherein hetero atoms such as oxygen, nitrogen, sulfur, silicon, phosphorus, boron, and the like, may optionally be present in the aryl group, in embodiments, having from about 6 to about 20 carbon atoms, or from about 6 to about 18 carbon atoms, although the numbers can be outside of these ranges, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms such as oxygen, nitrogen, sulfur, silicon, phosphorus, boron, and the like, may optionally be present in either the aryl portion or the alkyl portion of the arylalkyl, in embodiments, having from about 6 to about 20 carbon atoms, or from about 6 to about 18 carbon atoms, although the numbers can be outside of these ranges, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms such as oxygen, nitrogen, sulfur, silicon, phosphorus, boron, and the like, may optionally be present in either the alkyl portion or the aryl portion of the alkylaryl group, in embodiments, having from about 6 to about 20 carbon atoms, or from about 6 to about 18 carbon atoms, although the numbers can be outside of these ranges, (vi) an alkoxy group, (vii) an aryloxy group, which can be substituted or unsubstituted aryloxy, and wherein hetero atoms such as oxygen, nitrogen, sulfur, silicon, phosphorus, boron, and the like, may optionally be present in the aryl portion of the aryloxy group, in embodiments, having from about 6 to about 20 carbon atoms, or from about 6 to about 18 carbon atoms, although the numbers can be outside of these ranges, (viii) an arylalkyloxy group, which can be substituted or unsubstituted arylalkyloxy, wherein the alkyl portion of the arylalkyloxy can be linear or branched, saturated or unsaturated, cyclic or acyclic, and substituted or unsubstituted, and wherein hetero atoms such as oxygen, nitrogen, sulfur, silicon, phosphorus, boron, and the like, may optionally be present in either the aryl portion or the alkyl portion of the arylalkyloxy group, in embodiments, having from about 6 to about 20 carbon atoms, or from about 6 to about 18 carbon atoms, although the numbers can be outside of these ranges, (ix) an alkylaryloxy group, which can be substituted or unsubstituted alkylaryloxy, wherein the alkyl portion of the alkylaryloxy can be linear or branched, saturated or unsaturated, cyclic or acyclic, and substituted or unsubstituted, and wherein hetero atoms such as oxygen, nitrogen, sulfur, silicon, phosphorus, boron, and the like, may optionally be present in either the alkyl portion or the aryl portion of the alkylaryloxy group, in embodiments, having from about 6 to about 20 carbon atoms, or from about 6 to about 18 carbon atoms, although the numbers can be outside of these ranges; and

wherein R⁵ is (i) an alkylene group, (wherein an alkylene group is defined as a divalent aliphatic group or alkyl group, including linear and branched, saturated and unsaturated, cyclic and acyclic, and substituted and unsubstituted alkylene groups, and wherein hetero atoms such as oxygen, nitrogen, sulfur, silicon, phosphorus, boron, and the like, may optionally be present in the alkylene group), in embodiments, having from about 1 to about 20 carbon atoms, or from about 1 to about 18 carbon atoms, although the numbers can be outside of these ranges; (ii) an arylene group, (wherein an arylene group is defined as a divalent aromatic or aryl group, including substituted and unsubstituted arylene groups, and wherein hetero atoms such as described above for the alkylene groups may optionally be present in the arylene group), in embodiments, having from about 6 to about 20 carbon atoms, or from about 6 to about 18 carbon atoms, although the numbers can be outside of these ranges; (iii) an arylalkylene group, (wherein an arylalkylene group is defined as a divalent arylalkyl group, including substituted and unsubstituted arylalkylene groups, wherein the alkyl portion of the arylalkylene group can be linear or branched, saturated or unsaturated, cyclic or acyclic, and substituted or unsubstituted, and wherein hetero atoms such as described above for the alkylene groups may optionally be present in either the aryl portion of the alkyl portion of the arylalkylene group), in embodiments, having from about 6 to about 20 carbon atoms, or from about 6 to about 18 carbon atoms, although the numbers can be outside of these ranges; or (iv) an alkylarylene group, (wherein an alkylarylene group is defined as a divalent alkylaryl group, including substituted and unsubstituted alkylarylene groups, wherein the alkyl portion of the alkylarylene group can be linear or branched, saturated or unsaturated, cyclic or acyclic, and substituted or unsubstituted, and wherein hetero atoms such as described above for the alkylene groups may optionally be present in either the aryl portion or the alkyl portion of the alkylarylene group), in embodiments, having from about 6 to about 20 carbon atoms, or from about 6 to about 18 carbon atoms, although the numbers can be outside of these ranges;

wherein the substituents on the substituted alkyl, aryl, alkylaryl, arylalkyl, alkoxy, aryloxy, alkylaryloxy, aryalkyloxy, alkylene, arylene, arylalkylene, and alkylarylene groups of R¹ through R⁵ can be, but are not limited to, the following groups: pyridine, pyridinium, ether, aldehyde, ketone, ester, amide, carbonyl, thiocarbonyl, sulfide, phosphine, phosphonium, phosphate, nitrile, mercapto, nitro, nitroso, acyl, acid anhydride, azide, azo, thiocyanato, carboxylate, urethane, urea, mixtures and combinations thereof, and the like, wherein two or more substituents can be joined together to form a ring.

In embodiments, R¹, R², R³, and R⁴ can be the same or different, and each of R¹, R², R³, and R⁴ are independently selected from (i) hydrogen, an (ii) an alkyl group having from about 1 to about 20 carbon atoms.

In one embodiment, R¹, and R³ are hydrogen and R² and R⁴ are methyl.

In embodiment, R⁵ is an alkylene, an arylene, an alkylarylene, or an arylalkylene group having from about 1 to about 20 carbon atoms.

In specific embodiments, R⁵ is a compound of the formula

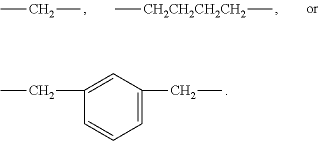

In specific embodiments, the charge transport molecule is of the formula

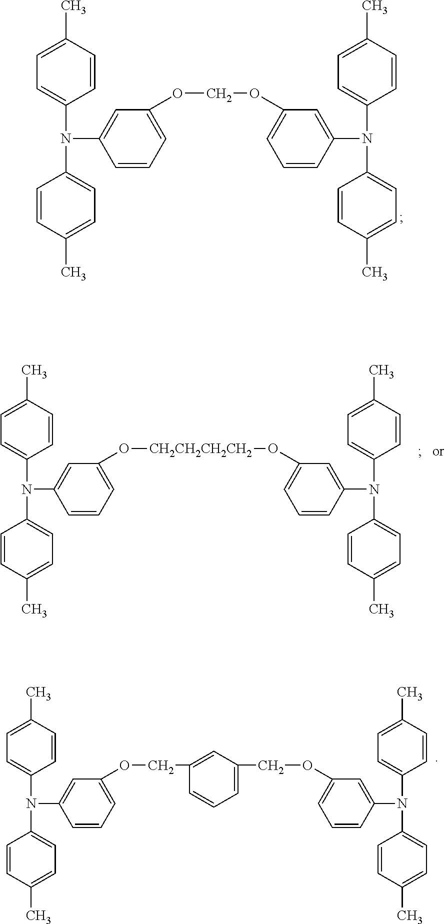

Compounds as disclosed herein can be prepared by any desired or effective method. In one specific embodiment, a process for preparing a charge transport compound herein comprises reacting about two molar equivalents of a hydroxy-functionalized triarylamine of the formula

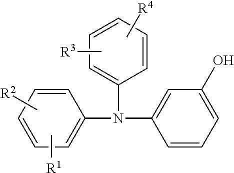

with about 1 molar equivalent of a dihalide of the formula

R⁵X₂

wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group;

wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group;

wherein X is a halogen, for example, wherein X is fluorine, chlorine, bromine, or iodine, and where, in specific embodiments, X is chlorine;

in an alkaline-water solution, at room temperature, typically from about 20 to about 26° C., although the temperature can be outside of these ranges; and optionally, treating the product to provide a purified product.

The alkaline-water solution can be any suitable or desired alkaline water solution. In embodiments, the alkaline-water solution comprises sodium hydroxide in water, potassium hydroxide in water, or a mixture or combination thereof. The alkaline-water solution can comprise any suitable or desired concentration. In embodiments, the alkaline-water solution can comprise from about 1 to about 60% aqueous solution. In specific embodiments, the alkaline-water solution comprises a 50% sodium hydroxide aqueous solution, a 50% potassium hydroxide aqueous solution, or a mixture or combination thereof.

In a specific embodiment, the process comprises providing the dihalide reactant from a halogen-organic solvent-containing waste stream. The waste stream can be a recycled solvent waste stream containing, for example, but not limited to, methylene chloride, which methylene chloride, or other halogen-organic, can be used as a reactant in the process herein.

In embodiments of the process herein, R¹, R², R³, and R⁴ can be the same or different, and wherein each of R¹, R², R³, and R⁴ are independently selected from (i) hydrogen, an (ii) an alkyl group having from about 1 to about 20 carbon atoms.

In a specific embodiment of the process herein, R¹, and R³ are hydrogen and wherein R² and R⁴ are methyl.

In another embodiment of the process herein, R⁵ is an alkylene, an arylene, an alkylarylene, or an arylalkylene group having from about 1 to about 20 carbon atoms.

In specific embodiments of the process herein, R⁵ is a compound of the formula

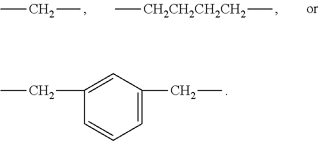

In one specific embodiment of the process herein, R⁵X₂ is methylene chloride of the formula

CH₂Cl₂.

The process herein can further optionally comprise treating the product by any suitable or desired process. For example, the product can be treated or purified by filtration, washing, crystallization, drying, or a combination thereof.

Charge transport molecules can be synthesized by the coupling reaction of 3-(N,N-ditolylamino)phenol [DTAP] with dihalides. The reaction can be run at room temperature in an alkaline-water solution. The yields are high, and the purification procedure is simple. In embodiments, the dihalides can be aliphatic or aromatic. In specific embodiments, recycled solvent waste containing dihalides, such as methylene chloride, can be used as (reactant) starting materials.

The present charge transport molecules have high charge transport mobility. In embodiments, the charge transport molecules herein have a high charge transport mobility, which is comparable with m-TBD (N,N′-diphenyl-N,N′-bis (3-methylphenyl)-[1,1′-biphenyl]-4,4′-diamine). Charge transport mobility can be considered an intrinsic property of a material and can be impacted by many factors such as testing field and loading level. In embodiments, m-TBD can have a charge transport mobility of about 1.0E-06 cm²V⁻¹S⁻¹.

For example, in embodiments, the charge transport molecules herein have a charge transport mobility of about 1.0E-05 cm²V⁻¹S⁻¹. In some embodiments, the charge transport molecules herein have a charge transport mobility of about 1.0E-05 cm²V⁻¹S⁻¹ when provided in a charge transport composition comprising a 50:50 weight ratio of charge transport molecule to polycarbonate binder.

In a specific embodiment, the process herein provides enables the conversion of organic halide waste into valuable charge transport materials.

Therefore, an environmentally friendly and convenient method to prepare charge transport molecules is provided. In embodiments, the reaction can occur in an alkaline-water solution at room temperature. In specific embodiments, purified or recovered waste streams of halo-organic solvents can be used as raw materials. The extraction and purification of the product are simple. In specific embodiments, a family of novel charge transport molecules can be synthesized by the coupling reaction of 3-(N,Nditolylamino) phenol [DTAP] with dihalides. The reactions can be run at room temperature in an alkaline-water solution. The yields are high, and can, in embodiments, be up to about 95 percent yield, and the purification procedure is simple.

The dihalides can be aliphatic or aromatic as described herein. Advantageously, even recycled solvent waste containing dihalides, such as methylene chloride, can be used as starting materials. The process herein provides the ability to convert organic halide waste into valuable charge transport materials. Further, the process herein provides alternate and comparable faster transport materials that are also very environmentally friendly as they can be prepared by reclaiming waste material.

Imaging members disclosed herein include, in embodiments, a substrate; thereover a charge generating layer; and thereover a charge transport layer comprising a compound of the formula

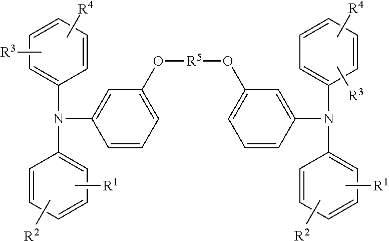

wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group; and wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group.

Methods for preparing an imaging member as disclosed herein include, in embodiments, a process comprising depositing a charge generating layer upon a substrate; and depositing a charge transport layer comprising a charge transport compound as described herein over the charge generating layer.

The charge transport molecule can be dissolved or dispersed in a polymer binder and then coated over the charge generating layer. The charge transport compound herein can be disposed in a polymer binder or any suitable material as is known, such as, for example, a polycarbonate or a polystyrene, in embodiments, Makrolon®.

The charge-transport component transports charge from the charge-generating layer to the surface of the photoreceptor.

Any suitable solvent or solvent system can be selected for embodiments herein in forming the layers. For example, the solvent system is selected in embodiments to assist in obtaining a stable dispersion of the foregoing components. Examples of suitable solvents include, but are not limited to, solvents selected from the group consisting of tetrahydrofuran, toluene, hexane, cyclohexane, cyclohexanone, methylene chloride, 1,1,2-trichloroethane, monochlorobenzene, and the like, and mixtures and combinations thereof. The total solid to total solvent can be selected in embodiments at an amount of from about 15:85 weight % to about 30:70 weight %, or from about 20:80 weight % to about 25:75 weight % although not limited.

Additional additives can be added as desired. For example, antioxidants, surfactants, or leveling agents can be included in the charge transport layer material as needed or desired. Any suitable antioxidant, leveling agent, or other additive can be included. In embodiments, a surfactant can be selected. Any suitable surfactant can be selected as desired. In embodiments, a trimethylsilyl end-capped polydimethyldiphenylsilane can be selected for the charge transport layer. For example, in embodiments, a trimethylsilyl end-capped polydimethyldiphenylsilane, DC 510®, available from Dow Corning, can be selected. Without wishing to be bound by theory, it is believed that this surfactant enhances the quality of charge transport layer coating and allows achievement of enhanced electrical and mechanical device characteristics. The surfactant can be added in any suitable amount, for example, in embodiments, an amount can be selected at from about 0.0001% to about 0.5%, or from about 0.0001% to about 0.1%, or about 0.005%, by weight based upon the total weight of the coating solution, although not limited to these amounts or ranges. Optionally, the surfactant material can be added to the charge generation layer.

The amounts of small molecule charge transport materials and binders, and ratios of components, can be selected as desired depending upon the final mobility desired for the devices. In selected embodiments, the charge transport layer contains the small molecule charge transport compound described herein and polymeric component selected at a weight ratio of from about 0:100 to about 90:10 small molecule charge transport compound to polymeric component.

The charge transport layer can be provided at any suitable thickness. For example, in embodiments, the charge transport layer has a thickness of from about 2 to about 35 micrometers.

Electrostatographic imaging members are well known in the art and may be prepared by various suitable techniques. Typically, a flexible or rigid substrate is provided having an electrically conductive surface. A charge generating layer is applied to the electrically conductive surface. A charge blocking layer may be applied to the electrically conductive surface prior to the application of the charge generating layer. If desired, an adhesive layer may be used between the charge blocking layer and the charge generating layer. The charge generation layer can be applied onto the blocking layer and a charge transport layer formed on the charge generation layer. In certain embodiments, the charge transport layer can be applied prior to the charge generation layer.

The supporting substrate can be selected to include a conductive metal substrate or a metallized substrate. While a metal substrate is substantially or completely metal, the substrate of a metallized substrate is made of a different material that has at least one layer of metal applied to at least one surface of the substrate. The material of the substrate of the metallized substrate can be any material for which a metal layer is capable of being applied. For instance, the substrate can be a synthetic material, such as a polymer. In various exemplary embodiments, a conductive substrate is, for example, at least one member selected from the group consisting of aluminum, aluminized or titanized polyethylene terephthalate belt (Mylar®).

Any metal or metal alloy can be selected for the metal or metallized substrate. Typical metals employed for this purpose include aluminum, zirconium, niobium, tantalum, vanadium, hafnium, titanium, nickel, stainless steel, chromium, tungsten, molybdenum, mixtures and combinations thereof, and the like. Useful metal alloys may contain two or more metals such as zirconium, niobium, tantalum, vanadium, hafnium, titanium, nickel, stainless steel, chromium, tungsten, molybdenum, mixtures and combinations thereof, and the like. Aluminum, such as mirror-finish aluminum, is selected in embodiments for both the metal substrate and the metal in the metallized substrate. All types of substrates may be used, including honed substrates, anodized substrates, bohmite-coated substrates and mirror substrates.

A metal substrate or metallized substrate can be selected. Examples of substrate layers selected for the present imaging members include opaque or substantially transparent materials, and may comprise any suitable material having the requisite mechanical properties. Thus, for example, the substrate can comprise a layer of insulating material including inorganic or organic polymeric materials, such as Mylar®, a commercially available polymer, Mylar® containing titanium, a layer of an organic or inorganic material having a semiconductive surface layer, such as indium tin oxide or aluminum arrange thereon, or a conductive material such as aluminum, chromium, nickel, brass or the like. The substrate may be flexible, seamless, or rigid, and may have a number of different configurations. For example, the substrate may comprise a plate, a cylindrical drum, a scroll, and endless flexible belt, or other configuration. In some situations, it may be desirable to provide an anticurl layer to the back of the substrate, such as when the substrate is a flexible organic polymeric material, such as for example polycarbonate materials, for example Makrolon® a commercially available material.

The thickness of the substrate layer depends on numerous factors, including strength desired and economical considerations. Thus, the substrate layer for a flexible belt can be of substantial thickness, for example, in embodiments, about 125 micrometers, or of minimum thickness, for example, in embodiments, less than 50 micrometers, provided there are no adverse effects on the final device. The surface of the substrate layer can be cleaned prior to coating to promote greater adhesion of the deposited coating. Cleaning may be effect, for example, by exposing the surface of the substrate layer to plasma discharge, ion bombardment, and the like.

Optionally, a hole blocking layer is applied, in embodiments, to the substrate. Generally, electron blocking layers for positively charged photoreceptors allow the photogenerated holes in the charge generating layer at the top of the photoreceptor to migrate toward the charge (hole) transport layer below and reach the bottom conductive layer during the electrophotographic imaging process. Thus, an electron blocking layer is normally not expected to block holes in positively charged photoreceptors such as photoreceptors coated with a charge generating layer over a charge (hole) transport layer. For negatively charged photoreceptors, any suitable hold blocking layer capable of forming an electronic barrier to holes between the adjacent photoconductive layer and the underlying substrate layer may be utilized. A hole blocking layer may comprise any suitable material. Typical hole blocking layers utilized for the negatively charged photoreceptors may include, for example, polyamides such as Luckamide® (a nylon-6 type material derived from methoxymethyl-substituted polyamide), hydroxyl alkyl methacrylates, nylons, gelatin, hydroxyl alkyl cellulose, organopolyphosphazenes, organosilanes, organotitanates, organozirconates, silicon oxides, zirconium oxides, and the like. In embodiments, the hole blocking layer comprises nitrogen containing siloxanes.

In embodiments, the hole blocking layer comprises gamma aminopropyl triethoxy silane.

The blocking layer, as with all layers herein, may be applied by any suitable technique such as, but not limited to, spraying dip coating, draw bar coating, gravure coating, silk screening, air knife coating, reverse roll coating, vacuum deposition, chemical treatment, and the like.

An adhesive layer may optionally be applied such as to the hole blocking layer. The adhesive layer may comprise any suitable material, for example, any suitable film forming polymer. Typical adhesive layer materials include, but are not limited to, for example, copolyester resins, polyarylates, polyurethanes, blends of resins, and the like. Any suitable solvent may be selected in embodiments to form an adhesive layer coating solution. Typical solvents include, but are not limited to, for example, tetrahydrofuran, toluene, hexane, cyclohexane, cyclohexanone, methylene chloride, 1,1,2-trichloroethane, monochlorobenzene, and mixtures thereof, and the like.

The charge-generating component converts light input into electron hole pairs. Examples of compounds suitable for use as the charge-generating component include vanadyl phthalocyanine, metal phthalocyanines (such as titanyl phthalocyanine, chlorogallium phthalocyanine, hydroxygallium phthalocyanine, and alkoxygallium phthalocyanine), metal-free phthalocyanines, benzimidazole perylene, amorphous selenium, trigonal selenium, selenium alloys (such as selenium-tellurium, selenium-tellurium arsenic, selenium arsenide), chlorogallium phthalocyanin, and mixtures and combinations thereof. In various exemplary embodiments, a photogenerating layer includes metal phthalocyanines and/or metal free phthalocyanines. In various exemplary embodiments, a photogenerating layer includes at least one phthalocyanine selected from the group consisting of titanyl phthalocyanines, perylenes, or hydroxygallium phthalocyanines. In various exemplary embodiments, a photogenerating layer includes Type V hydroxygallium phthalocyanine.

The charge generating layer may comprise in embodiments single or multiple layers comprising inorganic or organic compositions and the like. Suitable polymeric film-forming binder materials for the charge generating layer and/or charge generating pigment include, but are not limited to, thermoplastic and thermosetting resins, such as polycarbonates, polyesters, polyamides, polyurethanes, polystyrenes, polyarylethers, polyarylsulfones, polybutadienes, polysulfones, polyethersulfones, polyethylenes, polypropylenes, polyimides, polymethylpentenes, polyphenylene sulfides, polyvinyl acetate, polysiloxanes, polyacrylates, polyvinyl acetals, amino resins, phenylene oxide resins, terephthalic acid resins, phenoxy resins, epoxy resins, phenolic resins, polystyrene and acrylonitrile copolymers, polyvinyl chloride, vinylchloride and vinyl acetate copolymers, acrylate copolymers, alkyd resins, cellulosic film formers, poly(amideimide), styrene-butadiene copolymers, vinylidinechloride-vinylchloride copolymers, vinylacetate-vinylidenechloride copolymers, vinylacetate-vinylidenechloride copolymers, styrene-alkyd resins, polyvinylcarbazole, and mixtures thereof.

The charge-generating component may also contain a photogenerating composition or pigment. The photogenerating composition or pigment may be present in the resinous binder composition in various amounts, ranging from about 5% by volume to about 90% by volume (the photogenerating pigment is dispersed in about 10% by volume to about 95% by volume of the resinous binder); or from about 20% by volume to about 30% by volume (the photogenerating pigment is dispersed in about 70% by volume to about 80% by volume of the resinous binder composition). In one embodiment, about 8 percent by volume of the photogenerating pigment is dispersed in about 92 percent by volume of the resinous binder composition. When the photogenerating component contains photoconductive compositions and/or pigments in the resinous binder material, the thickness of the layer typically ranges from about 0.1 μm to about 5.0 μm, or from about 0.3 μm to about 3 μm. The photogenerating layer thickness is often related to binder content, for example, higher binder content compositions typically require thicker layers for photogeneration. Thicknesses outside these ranges may also be selected.

The thickness of the imaging device typically ranges from about 2 μm to about 100 μm; from about 5 μm to about 50 μm, or from about 10 μm to about 30 μm. The thickness of each layer will depend on how many components are contained in that layer, how much of each component is desired in the layer, and other factors familiar to those in the art.

As with the various other layers described herein, the photogenerating layer can be applied to underlying layers by any desired or suitable method. Any suitable technique may be employed to mix and thereafter apply the photogenerating layer coating mixture with typical application techniques including, but not being limited to, spraying, dip coating, roll coating, wire wound rod coating, and the like. Drying, as with the other layers herein, can be effect by any suitable technique, such as, but not limited to, over drying, infrared radiation drying, air drying, and the like.

Optionally, an overcoat layer can be employed to improve resistance of the photoreceptor to abrasion. An optional anticurl back coating may further be applied to the surface of the substrate opposite to that bearing the photoconductive layer to provide flatness and/or abrasion resistance where a web configuration photoreceptor is desired. These overcoating and anticurl back coating layers are well known in the art, and can comprise for example thermoplastic organic polymers or inorganic polymers that are electrically insulating or slightly semiconductive. In embodiments, overcoatings are continuous and have a thickness of less than about 10 microns, although the thickness can be outside this range. The thickness of anticurl backing layers is selected in embodiments sufficient to balance substantially the total forces of the layer or layers on the opposite side of the substrate layer. In embodiments, the second Makrolon®/TPD transport layer can be considered as a thick overcoat layer.

Various exemplary embodiments encompassed herein include a method of imaging which includes generating an electrostatic latent image on an imaging member, developing a latent image, and transferring the developed electrostatic image to a suitable substrate.

Further embodiments encompassed within the present disclosure include methods of imaging and printing with the photoresponsive devices illustrated herein. Various exemplary embodiments include methods including forming an electrostatic latent image on an imaging member; developing the image with a toner composition including, for example, at least one thermoplastic resin, at least one colorant, such as pigment, at least one charge additive, and at least one surface additive; transferring the image to a necessary member, such as, for example any suitable substrate, such as, for example, paper; and permanently affixing the image thereto. In various exemplary embodiments in which the embodiment is used in a printing mode, various exemplary imaging methods include forming an electrostatic latent image on an imaging member by use of a laser device or image bar; developing the image with a toner composition including, for example, at least one thermoplastic resin, at least one colorant, such as pigment, at least one charge additive, and at least one surface additive; transferring the image to a necessary member, such as, for example any suitable substrate, such as, for example, paper; and permanently affixing the image thereto.

In a selected embodiment, an image forming apparatus for forming images on a recording medium comprises a) a photoreceptor member having a charge retentive surface to receive an electrostatic latent image thereon, wherein said photoreceptor member comprises a metal or metallized substrate, a charge generating layer, and a charge transport layer comprising charge transport materials dispersed therein; b) a development component to apply a developer material to said charge-retentive surface to develop said electrostatic latent image to form a developed image on said charge-retentive surface; c) a transfer component for transferring said developed image from said charge-retentive surface to another member or a copy substrate; and d) a fusing member to fuse said developed image to said copy substrate.

### EXAMPLES

The following Examples are being submitted to further define various species of the present disclosure. These Examples are intended to be illustrative only and are not intended to limit the scope of the present disclosure. Also, parts and percentages are by weight unless otherwise indicated.

### Example 1

A charge transport molecule of the formula

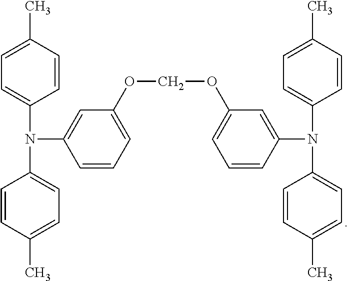

(Product I) was prepared as follows. A mixture of 3-(N,N-ditolylamino)phenol (DTAP, prepared as described in U.S. Pat. Nos. 5,976,744 and 6,099,996, each of which is hereby incorporated by reference herein in its entirety) (14.5 grams, 50 millimoles), 50% NaOH aqueous solution (15 milliliters), tetrabutylammonium hydrogen sulfate (available from Sigma-Aldrich, Inc.) (3.4 grams, 10 millimoles) and methylene chloride (available from Sigma-Aldrich, Inc.) (30 milliliters) was stirred at room temperature for 15 hours. Thereafter, the solvents were removed using a rotavapor (Rotavapor® R-210/R-215 available from Buchi Laboratory Equipment). The remaining yellow paste was poured into 150 milliliters of deionized water with vigorous stirring. The solid was collected by filtration, and washed three times with methanol (50 milliliters each wash). After vacuum drying at 60° C., Product I was obtained as white crystals (14.0 grams) in 94% yield. The molecular structure of Product I was confirmed by NMR and FT-IR. Further purification was carried out by recrystallization from ethyl acetate.

### Example 2

A charge transport molecule of the formula

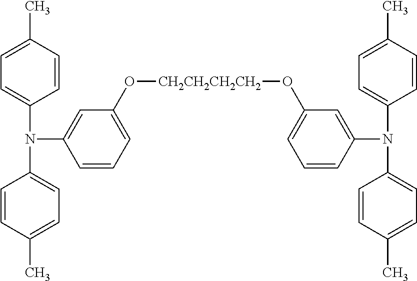

(Product II) was prepared as follows. A mixture of 3-(N,N-ditolylamino)phenol (DTAP, prepared as described in U.S. Pat. Nos. 5,976,744 and 6,099,996, each of which is hereby incorporated by reference herein in its entirety) (5.8 grams, 20 millimoles), 50% NaOH aqueous solution (30 milliliters), tetrabutylammonium hydrogen sulfate (available from Sigma-Aldrich, Inc.) (3.4 grams, 10 millimoles) and 1.4-dibromobutane (available from Sigma-Aldrich, Inc.) (2.15 grams, 10 millimoles) was stirred vigorously at 40° C. for 18 hours. The viscous yellow solution was poured into 150 milliliters of deionized water with vigorous stirring. The solid was collected by filtration, and washed three times with methanol (50 milliliters for each wash) and then three times with water (50 milliliters for each wash). After drying in a vacuum oven at 60° C., Product II was obtained as white crystals (4.9 grams) in 78% yield. NMR and FT-IR were used to confirm the molecular structure of Product II.

### Example 3

A charge transport molecule of the formula

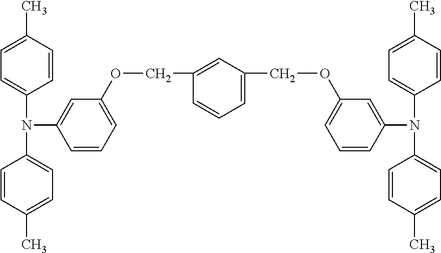

(Product II) was prepared as follows. A mixture of 3-(N,N-ditolylamino)phenol (DTAP, prepared as described in U.S. Pat. Nos. 5,976,744 and 6,099,996, each of which is hereby incorporated by reference herein in its entirety) (5.8 grams, 50 millimoles), 50% KOH aqueous solution (15 milliliters), benzyltrimethylammonium chloride (available from Sigma-Aldrich, Inc.) (1.9 grams, 10 millimoles), α,α′-dibromo-m-xylene (available from Sigma-Aldrich, Inc.) (97% purity, 2.64 grams, 10 millimoles) and toluene (30 milliliters) was stirred at room temperature for 15 hours. Thereafter, the solvents were taken off using a rotary evaporator. The remaining yellow paste was poured into 150 milliliters of deionized water with vigorous stirring. The solid was collected by filtration, and washed with three 50 milliliters portions of methanol. After drying in a vacuum oven at 60° C., Product III was obtained as white crystals (5.7 grams) in 84% yield. The molecular structure of Product III was confirmed using NMR and FT-IR. Further purification was performed by recrystallization from acetone.

### Comparative Example 4

A charge transport molecule, N,N′-diphenyl-N,N′-bis(3-hydroxyphenyl)-[1,1′-biphenyl]-4,4′-diamine (DHTBD) of the formula

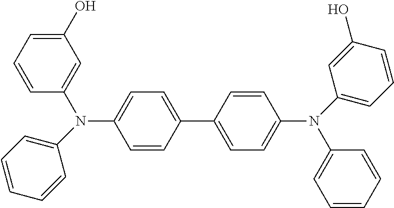

was prepared as described in U.S. Pat. No. 4,806,443, which is hereby incorporated by reference herein in its entirety, Examples I and II. The DHTBD has a mobility of about 2.88E-09 cm²V⁻¹S⁻¹.

### Example 5

### Preparation of Imaging Members Up Through Charge Generating Layer

An electrophotographic imaging member web stock was prepared by providing a 0.02 micrometer thick titanium layer coated on a biaxially oriented polyethylene naphthalate substrate (KADALEX™, available from ICI Americas, Inc.) having a thickness of 3.5 mils (89 micrometers) and applying thereto, using a gravure coating technique or a die extrusion coating technique, a solution containing 10 grams gamma aminopropyltriethoxysilane, 10.1 grams distilled water, 3 grams acetic acid, 684.8 grams of 200 proof denatured alcohol and 200 grams heptane. This layer was then allowed to dry for 5 minutes at 135° C. in a forced air oven. The resulting blocking layer had an average dry thickness of 0.05 micrometer measured with an ellipsometer.

An adhesive interface layer was then prepared by applying with an extrusion process to the blocking layer a wet coating containing 5 percent by weight based on the total weight of the solution of polyester adhesive (Ardel®) in a 70:30 volume ratio mixture of tetrahydrofuran:cyclohexanone. The adhesive interface layer was allowed to dry for 5 minutes at 135° C. in a forced air oven. The resulting adhesive interface layer had a dry thickness of 0.065 micrometer

The adhesive interface layer was thereafter coated with a charge generating layer. The charge generating layer dispersion was prepared by introducing 0.45 grams of LUPILON® 200 (PC-Z 200) available from Mitsubishi Gas Chemical Corp. and 50 ml of tetrahydrofuran into a 4 oz. glass bottle. To this solution was added 2.4 grams of hydroxygallium phthalocyanine (OHGaPc) and 300 grams of ⅛ inch (3.2 millimeter) diameter stainless steel shot. This mixture was then placed on a ball mill for 6 to 8 hours. Subsequently, 2.25 grams of PC-Z 200 was dissolved in 46.1 gm of tetrahydrofuran, then added to this OHGaPc slurry. This slurry was then placed on a shaker for 10 minutes. The resulting slurry was, thereafter, coated onto the adhesive interface by an extrusion application process to form a layer having a wet thickness of 0.25 mil. A strip about 10 mm wide along one edge of the substrate web bearing the blocking layer and the adhesive layer was deliberately left uncoated by any of the charge generating layer material to facilitate adequate electrical contact by the ground strip layer that is applied later. This charge generating layer was dried at 135° C. for 5 minutes in a forced air oven to form a dry charge generating layer having a thickness of 0.4 micrometer layer.

### Example 6

### Preparation of Imaging Members Up Through Charge Transport Layer

Charge transport layer coating solutions were then prepared for Examples 1 through Comparative Example 4. To form each of the charge transport layer solutions, about 3.6 grams of each of the charge transport material of Examples 1 through Comparative Example 4 were combined with about 3.6 grams of poly(4,4′-diphenyl-1,1′-cyclohexane carbonate)-400, with a weight average molecular weight of 40,000, about 22.5 grams of tetrahydrofuran and about 7.5 grams of toluene in a 60 milliliter brown bottle. After mixing on a rolling mill at from about 20 to about 20° C. for about 15 hours, the four charge transports solutions of Examples 1 through Comparative Example 4 were ready for coating onto the photoreceptor structure of Example 5. The devices had a total thickness of about 30 micrometers. See also U.S. Pat. No. 6,677,090, which is hereby incorporated by reference herein in its entirety, for a description of photoreceptor device fabrication.

Mobility can be determined by any suitable or desired method. Charge Transport Mobility for exemplary charge transport materials herein was determined as follows. Devices were furnished with ½ inch circular semitransparent gold electrode on the top surface to conduct time of flight measurements (TOF). Charges were injected from the charge generating layer through flash exposure for the gold electrodes biased at various set negative potentials. From the resulting transient currents, the transit time of the leading edge of the charges were measured. From these transient times for the different bias potentials, the mobility as a function of electric field was computed.

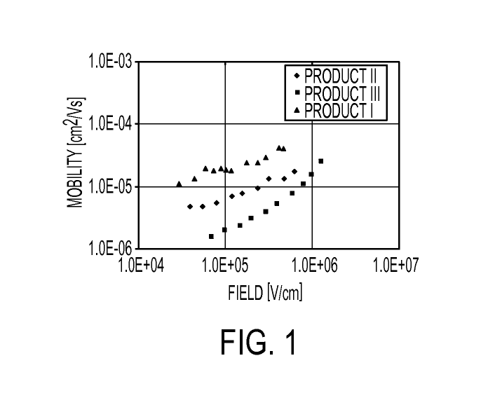

FIG. 1 provides charge mobility measurements for the charge transport materials of Examples 1-3, wherein mobility is shown on the y-axis as cm²/Vs versus field, shown on the x-axis (V/cm).

FIG. 1 shows that the present charge transport molecules have very high charge transport mobility. In embodiments, the charge transport molecules herein have a charge transport mobility of about 1.0E-05 cm²V⁻¹S⁻¹ with about 50/50 by weight ratio of charge transport molecule to polycarbonate binder.

As seen in FIG. 1, the mobility of the charge transport molecule of Product I herein was higher than the mobility of m-TBD (N,N-diphenyl-N,N-bis(3-methylphenyl)-[1,1′-biphenyl]-4,4′-diamine), which typically has a mobility of 1.0E-06 cm²V⁻¹S⁻¹.

It will be appreciated that various of the above-disclosed and other features and functions, or alternatives thereof, may be desirably combined into many other different systems or applications. Also that various presently unforeseen or unanticipated alternatives, modifications, variations or improvements therein may be subsequently made by those skilled in the art which are also intended to be encompassed by the following claims. Unless specifically recited in a claim, steps or components of claims should not be implied or imported from the specification or any other claims as to any particular order, number, position, size, shape, angle, color, or material.

1. A compound of the formula 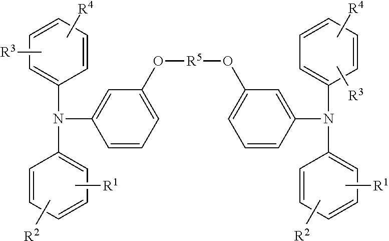
wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group; and
wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group.

2. The compound of claim 1, wherein R¹, R², R³, and R⁴ can be the same or different, and wherein each of R¹, R², R³, and R⁴ are independently selected from (i) hydrogen, an (ii) an alkyl group having from about 1 to about 20 carbon atoms.

3. The compound of claim 1, wherein R¹, and R³ are hydrogen and wherein R² and R⁴ are methyl.

4. The compound of claim 1, wherein R⁵ is an alkylene, an arylene, an alkylarylene, or an arylalkylene group having from about 1 to about 20 carbon atoms.

5. The compound of claim 1, wherein R⁵ is a compound of the formula

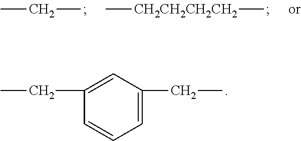

6. The compound of claim 1, of the formula

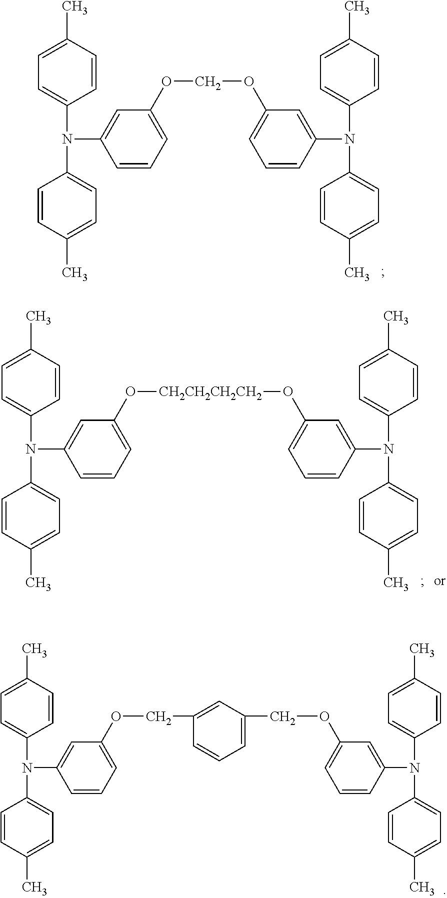

7. The compound of claim 1, wherein the compound has a charge transport mobility of about 1.0E-05 cm²V⁻¹S⁻¹ when provided in a charge transport composition comprising a 50:50 weight ratio of charge transport molecule to polycarbonate binder.

8. A process for preparing a charge transport compound comprising:
contacting a hydroxy-functionalized triarylamine of the formula 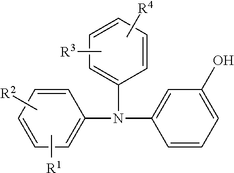
with a dihalide of the formula
R⁵X₂
wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group;
wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group;
wherein X is a halogen;
in an alkaline-water solution at room temperature; and
optionally, treating the product to provide a purified product.

9. The process of claim 8, further comprising:
providing the dihalide from a halogen-organic solvent-containing waste stream.

10. The process of claim 8, wherein R¹, R², R³, and R⁴ can be the same or different, and wherein each of R¹, R², R³, and R⁴ are independently selected from (i) hydrogen, an (ii) an alkyl group having from about 1 to about 20 carbon atoms.

11. The process of claim 8, wherein R¹, and R³ are hydrogen and wherein R² and R⁴ are methyl.

12. The process of claim 8, wherein R⁵ is an alkylene, an arylene, an alkylarylene, or an arylalkylene group having from about 1 to about 20 carbon atoms.

13. The process of claim 8, wherein R⁵ is a compound of the formula

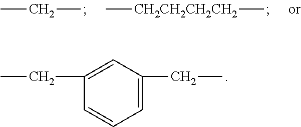

14. The process of claim 8, wherein X is fluorine, chlorine, bromine, or iodine.

15. The process of claim 8, wherein R⁵X₂ is methylene chloride of the formula CH₂Cl₂.

16. The process of claim 8, wherein treating the product comprises filtration, washing, crystallization, drying, or a combination thereof.

17. The process of claim 8, wherein the charge transport compound has a charge transport mobility of about 1.0E-05 cm²V⁻¹S⁻¹ when provided in a charge transport composition comprising a 50:50 weight ratio of charge transport molecule to polycarbonate binder.

18. An imaging member comprising:
a substrate;
thereover a charge generating layer; and
thereover a charge transport layer comprising a compound of the formula 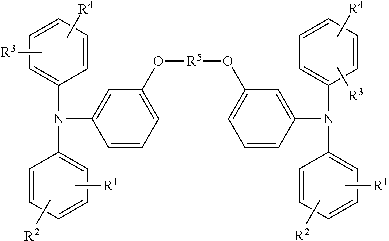
wherein R¹, R², R³, and R⁴ can be the same or different, and wherein each of R¹, R², R³, and R⁴ are independently selected from (i) hydrogen, (ii) an alkyl group, which can be linear or branched, saturated or unsaturated, cyclic or acyclic, substituted or unsubstituted alkyl, and wherein hetero atoms may optionally be present in the alky group, (iii) an aryl group, which can be substituted or unsubstituted aryl, and wherein hetero atoms may optionally be present in the aryl group, (iv) an arylalkyl group, which can be substituted or unsubstituted arylalkyl, wherein the alkyl portion of the arylalkyl can be linear or branched, saturated or unsaturated, cyclic or acyclic, and substituted or unsubstituted, and wherein hetero atoms may optionally be present in either the aryl portion or the alkyl portion of the arylalkyl, (v) an alkylaryl group, which can be substituted or unsubstituted alkylaryl, wherein the alkyl portion of the alkylaryl can be linear or branched, saturated or unsaturated, cyclic or acyclic, and substituted or unsubstituted, and wherein hetero atoms may optionally be present in either the alkyl portion or the aryl portion of the alkylaryl group, (vi) an alkoxy group, (vii) an aryloxy group, (viii) an arylalkyloxy group, (ix) an alkylaryloxy group; and
wherein R⁵ is (i) an alkylene group, which can be linear or branched, saturated or unsaturated, cyclic or acyclic, substituted or unsubstituted alkylene, and wherein hetero atoms may optionally be present in the alkylene group; (ii) an arylene group, which can be substituted or unsubstituted arylene, and wherein hetero atoms may optionally be present in the arylene group; (iii) an arylalkylene group, which can be substituted or unsubstituted arylalkylene, wherein the alkyl portion of the arylalkylene group can be linear or branched, saturated or unsaturated, cyclic or acyclic, and substituted or unsubstituted, and wherein hetero atoms may optionally be present in either the aryl portion or the alkyl portion of the arylalkylene group; or (iv) an alkylarylene group, which can be substituted or unsubstituted alkylarylene groups, wherein the alkyl portion of the alkylarylene group can be linear or branched, saturated or unsaturated, cyclic or acyclic, and substituted or unsubstituted, and wherein hetero atoms may optionally be present in either the alkyl portion or the aryl portion of the alkylarylene group.

19. An image forming apparatus for forming images on a recording medium comprising the imaging member of claim 18, the apparatus comprising:
a) a photoreceptor member having a charge retentive surface to receive an electrostatic latent image thereon, wherein said photoreceptor member comprises the imaging member of claim 18;
b) a development component to apply a developer material to said charge-retentive surface to develop said electrostatic latent image to form a developed image on said charge-retentive surface;
c) a transfer component for transferring said developed image from said charge-retentive surface to another member or a copy substrate; and
d) a fusing member to fuse said developed image to said copy substrate.

20. The imaging member of claim 18, wherein the charge transport compound is of the formula [chemical structure]
